(19) United States
(12) Patent Application Publication
(10) Pub. No.: US 2012/0191019 A1
(43) Pub. Date: Jul. 26, 2012

(54) APPARATUS AND METHODS FOR NON-INVASIVE BODY CONTOURING

(75) Inventors: Charles S. Desilets, Mukilteo, WA (US); Patrick J. Martin, Seattle, WA (US); Jens U. Quistgaard, Seattle, WA (US); Lawrence L. Kunz, Greenbank, WA (US)

(73) Assignee: Medicis Technologies Corporation, Bothell, WA (US)

(21) Appl. No.: 13/189,179

(22) Filed: Jul. 22, 2011

Related U.S. Application Data
(60) Provisional application No. 61/367,400, filed on Jul. 24, 2010.

(51) Int. Cl. A61N 7/00

(52) U.S. Cl. 601/2

(57) ABSTRACT

Methods and devices for applying high intensity focused ultrasound to modulate collagen in animal tissue (such as to disrupt collagen fibrils in a mammal, such as a human), particularly to enhance the aesthetic appearance of skin, and/or to otherwise improve skin conditions.

### CROSS-REFERENCES TO RELATED APPLICATIONS

This application claims the benefit of provisional application No. 61/367,400 (Attorney Docket No. 87704-789988 (008100US)), filed on Jul. 24, 2010, the full disclosure of which is incorporated herein by reference.

### FIELD

The present invention relates to using high intensity focused ultrasound energy to modify collagen and/or adipose tissue using non-invasive techniques.

### BACKGROUND

Application of a number of energy mediated techniques to improve skin appearance (e.g., to maintain a youthful skin appearance) have been proposed, described, and/or applied. Such methods have either been applied in seeking, or described as being able, to improve skin condition and skin tone such as by improving conditions associated with cellulite, photoaging, wrinkles, or loose skin.

High intensity focused ultrasound energy has been used as a method to improve aesthetic appearance, most notably in the destruction of adipose tissue and has been proposed as a mechanism for modulation of collagen, as described for example in commonly owned U.S. Pat. Nos. 7,258,674; 7,273,459; 7,766,848; 7,311,679; 7,532,201; 7,695,437 7,652,411; and U.S. Patent Publication Nos. 2005/0154295; 2005/0154431; 2007/0055156; 2005/0154314; 2008/0200813; 2006/0122509; 2009/0318837; 2007/0238994; 2008/0243035; 2008/0243003; 2009/0240146; 2009/0171252; 2009/0248578; and 2010/0042019, the full disclosures of which are incorporated herein by reference. While the disruption of collagen and/or adipose tissue is advantageous in many if not all of these procedures, consistently providing such disruption non-invasively has proven to be difficult.

### BRIEF SUMMARY

The methods and systems of the present invention are founded at least partially on the inventors' discovery and identification of precise, effective, and/or alternative methods of non-invasively applying high intensity focused ultrasound to modulate collagen in animal tissue (such as to disrupt collagen fibrils in a mammal, such as a human), particularly to enhance the aesthetic appearance of skin, and/or to otherwise improve skin conditions (such as enhancing skin tightening and/or enhancing other aspects of skin tone and/or appearance). The various aspects of the invention described herein provide such methods and other advantageous techniques for improving aesthetic appearance in mammalian skin. In another sense, the present invention provides systems and methods that improve the appearance of a mammal's, such as a human patient's, skin tone.

In one exemplary embodiment, a method of non-invasively disrupting collagen fibrils in animal tissue comprises positioning or otherwise presenting a high intensity focused ultrasound (HIFU) transducer on a skin surface of a patient, coupling the HIFU transducer to the skin surface to maximize energy coupling from the HIFU transducer through the skin surface, and directing the HIFU transducer to deposit an average fluence (“EF”) value of at least 35 J/cm² such that collagen fibrils below the dermis of the patient are detectably disrupted. In a more particular aspect of the invention, the collagen fibrils are at least about 30% disrupted by application of such a method.

In still other aspects, the fibrils that are disrupted are located between the dermis and the campers fascia of the patient. The fibrils may be allowed to restore and strengthen through a wound healing process, or the patient may be regularly massaged during a typically wound healing process to prevent restoration.

In another embodiment, a method of non-invasively contracting collagen fibrils in animal tissue comprises positioning or otherwise presenting a HIFU transducer on a skin surface of a patient, coupling the HIFU transducer to the skin surface to maximize energy coupling from the HIFU transducer through the skin surface and activating the HIFU transducer to deliver or deposit an average EF value of at least 35 J/cm² such that collagen fibrils below a dermis of the patient are at reduced at least 30% in length.

In still another embodiment, a method of measuring skin tone improvement comprises locating a region of human tissue beneath a skin surface and determining an initial condition of or for the skin surface. The method then includes a step of injuring the region of human tissue using a HIFU apparatus such that a volume of disrupted adipose cells and denatured and/or disrupted collagen fibrils are produced. The method then provides for allowing the injured region of human tissue to resolve and remodel and determining one or more post operative condition(s) for the skin surface after the injury to the region of human tissue has been created.

### BRIEF DESCRIPTION OF THE DRAWINGS

FIG. 1 shows a representation of a cross section of human tissue of a female human that exhibits cellulite.

FIG. 2 shows a representation of a cross section of the human tissue of FIG. 1, showing targeting disruption via HIFU treatments.

FIG. 3 shows a representation of a cross section of the human tissue of FIG. 2, after the targeted disruption.

FIG. 4 shows a representation of a cross section of the human tissue of FIG. 3, after wound process has occurred.

FIG. 5 shows a method of disrupting collagen fibrils F in accordance with additional embodiments.

FIG. 6 shows a HIFU device that may be used in embodiments.

FIG. 7 shows a computer system that may be used for directing the HIFU device of FIG. 6.

FIG. 8 shows modules that may be utilized by the computer system of FIG. 7 in accordance with embodiments.

### DETAILED DESCRIPTION

The nature of the systems and apparatus described herein are those of electronic devices. There are electrical signals being sent from various parts or sub systems to other parts and sub systems, as well as electrical power sent to those same parts (components) and sub systems. The communication between any component with any other component is referred to herein as “electrical communication.” Electrical communication may occur via signals or power, used to direct, sense, control or simply turn on a component. The passage of electrons through any intended conduit for electrons, regardless of voltage, amperage or wattage is considered electrical communication. Electrical communication further includes signals sent and received by wireless systems or methods if incorporated to any part of the disclosure herein.

“Collagen Fibril” refers to the collagen-containing material found in adipose tissue or sub dermal regions where collagen concentration tends to be sparse and used by the body as a lattice connective tissue rather than a major structural component (as contrasted with regions like the nose, ears, skin, tendons, and the like). “Contraction of collagen fibrils” may be effected using energy, such as thermal energy, to force the collagen to contract. Contraction typically occurs through denaturing the collagen protein, which may cause collagen fibrils to shorten lengthwise immediately and also initiate a wound healing process that over a period of weeks or months results in the contraction of a newly grown extra-cellular collagen matrix.

In an embodiment, energy mediated skin effects are produced using high intensity focused ultrasound (HIFU) energy. “HIFU” is ultrasound energy producing sufficient intensity (power per unit area) through focusing to modify tissue at the focal zone of the transducer. Modification is permanent and achieved by producing an average fluence (energy per units area or EF) in the range of 35-460 J/cm² and peak intensities in the range of 5-30 kW/cm².

Fluence is the total energy delivered to a treatment area (expressed here in Joules per square centimeter) and is independent of the means of delivery, i.e., whether the energy is focused or not. It is also independent of the length of time over which the energy is delivered. The term “Energy Flux” was used in earlier patent disclosures for this parameter, but the term has been changed in this disclosure to “fluence” to be in accord with common scientific nomenclature.

Ultrasonic intensity (power per unit area, expressed here in units of Watts per square centimeter), along with fluence, are the key parameters for determining the amount of temperature rise in a load medium, including human tissue. Intensity is proportional to the square of the ultrasonic pressure. In general, the heating rate, or the change in temperature per unit time, is proportional to the intensity. Focusing ultrasound dramatically increases the intensity, hence, the heating rate at the focus, since almost all the ultrasonic power is concentrated into the small focal area. With a constant focusing geometry and ultrasonic frequency, the intensity is proportional to the total output ultrasonic power.

HIFU fluence can be delivered in a multitude of ways, such as in series of short, high intensity bursts normally spaced periodically in time, or continuously at a lower intensity level. However, the bioeffects associated with short, high peak intensity bursts are more pronounced than with continuous lower intensity focused ultrasound as the result of nonlinearities arising from high intensity bursts. This is true even if the average source intensity is the same between the burst and continuous treatment parameter sets. These nonlinearities result in harmonic generation at even frequency multiples of the emitted frequency, especially in the high intensity focal zone, which can greatly increase the heating rate, and thus decrease the treatment time. The peak intensities tested in this product were simulated to range from 5 W/cm² to 30 kW/cm².

Fluence is determined by the following relationship for a moving transducer:

EF=[(p)×(l/v)×(dc)×(nl)]/(sa)

wherein

p=peak power,

l=line length,

v=velocity,

dc=duty cycle,

nl=number of lines

and

sa=scanned area.

Alternatively for a treatment program where the transducer is not moving between therapy applications, the EF can be calculated using the following modified EF equation.

EF=[(p)×(t)×(dc)×(ns)]/(sa)

wherein

p=peak power,

t=on-time per lesion,

dc=duty cycle,

ns=number of lesions,

and

sa=scanned area.

Fluence need not be constant, but may vary with higher peak intensity pulses and low to no energy output between pulses. Modification of tissue may not be restricted to the focal zone, ancillary damage or modification may occur depending on the length of time the focal zone of the transducer is positioned over a certain location. Slow or non-movement of the focal zone can cause additional energy to be deposited into the target tissue, and this effect may be intended under certain conditions.

Treatment regimens that utilize different operating parameters may be designed to produce various different effects in animal or human tissue. Reference is made to both animal and human tissue herein to illustrate actual clinical data on the various methods described herein: these methods have been tried on both animal and human patients.

In embodiments, a method of and device for non-invasively disrupting collagen fibrils in animal tissue are provided. The method comprises presenting of a HIFU apparatus on a skin surface, coupling the HIFU apparatus to the skin surface to maximize energy coupling from the HIFU apparatus through the skin surface and activating the HIFU apparatus to deposit an average fluence (EF) value of at least 35 J/cm² such that collagen fibrils are at least 30% disrupted. The device includes a HIFU device that is programmed, or that can be programmed, to provide such a function.

The collagen fibrils within the animal tissue are generally within or very close to the focal zone of a HIFU transducer incorporated into the apparatus, although it is not necessarily the case that the HIFU transducer is within an apparatus. In an embodiment the HIFU transducer may be “naked” (i.e., without any structural elements surrounding it) or essentially “naked.”

The peak intensity and average fluence of the HIFU apparatus can be used to produce the disruption of collagen fibrils. Disruption is the severing of collagen fibrils so that a single “strand” of collagen prior to treatment, is broken into two or more pieces after treatment. The peak fluence may be the same as the average fluence, or the peak fluence may be higher. In an aspect of the invention, the average fluence may vary between 35 and 460 J/cm². In another aspect the average fluence is 100 J/cm². In still another aspect the average fluence is 174 J/cm², and in yet another aspect the average fluence is 265 J/cm². Average fluence variations such as these embodiments, and other embodiments as may be determined without undue experimentation allow an operator to optimize the average fluence and peak intensity values to achieve a desired clinical result in a target tissue.

In various embodiments, the target tissue(s) may be human adipose tissue, human subcutaneous tissue and/or human dermis tissue. Aspects herein, however, are directed to disruption of collagen fibrils below the dermis tissue. Applicant possesses data showing the disruption of collagen fibrils in both humans and animal patient models. While data exists for both models (human and porcine) the larger body of data has been produced in the porcine model. A like response in human tissue can be inferred based on the porcine model which is widely accepted as the best animal model for this kind of study. Because the physiology of the porcine model is well recognized as the closest to human tissue for these applications, applicant has every reasonable belief that the results produced from the porcine model will carry over in human tissue.

A variety of factors can effect the percent efficiency of the HIFU energy in tissue. Depending on the EF value, and the type of tissue targeted, as well as the morphology of the tissue, the level of disruption of the collagen fibrils may range from 30% to over 85%. Thus in an embodiment, the disruption of collagen fibrils is at least 30%. In another embodiment, the disruption is 50%. In still another aspect, the disruption is 65%. Under ideal conditions, the level of collagen fibrils disruption may be 75 to 80%. In addition to disrupting collagen fibrils, denaturing collagen fibrils and/or disruption of adipose tissue may also result. Enhanced results may be achieved by moving the HIFU apparatus over the skin surface while the HIFU transducer is broadcasting ultrasound energy. Such movement increases the likelihood that the focal zone of the HIFU apparatus will come into contact with targeted collagen fibrils.

In another alternative embodiment, the focused ultrasound energy may produce a superheated pocket of liquid (such as water or plasma) that expands suddenly, and destroys collagen fibrils like a plastic string caught in an explosion. The sudden disruption of a pocket of tissue or interstitial fluid can produce vacuoles where the ultrasound energy causes sudden and disruptive mechanical damage to the local cellular and collagen structures.

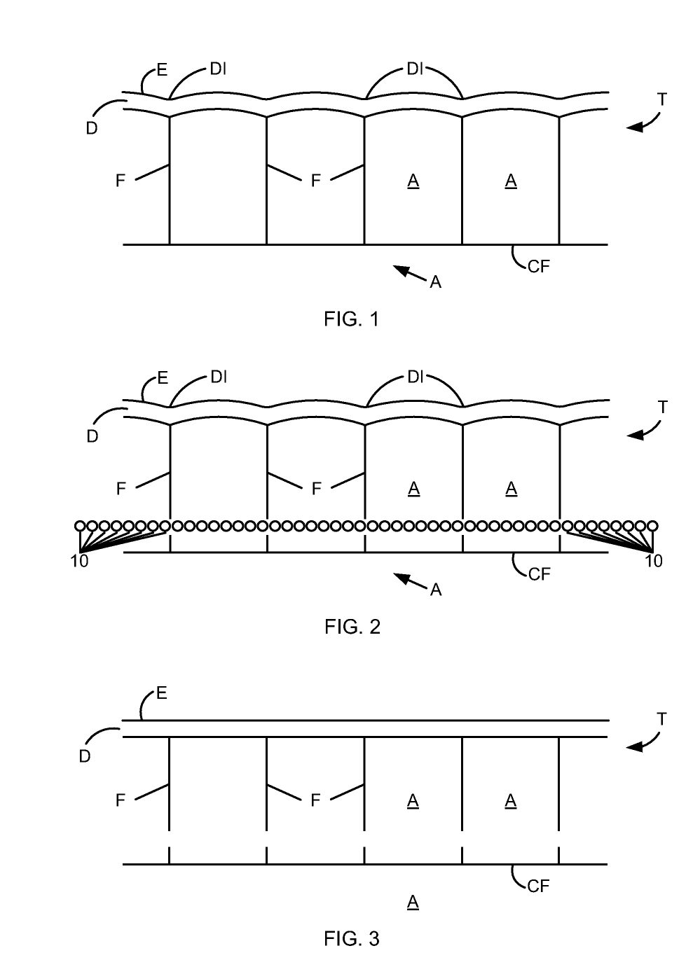

An example of a disruption process is shown in FIGS. 1 and 2. FIG. 1 shows a representation of a cross section of human tissue T of a human that exhibits cellulite. The tissue T is shown prior to treatment. Collagen fibrils F extend between the dermis D and the campers fascia CF in the patient. Campers fascia is a thick superficial layer of the anterior abdominal wall, but as used herein means any similar layer in other parts of the body. Adipose tissue A is located below the dermis, and above and below the campers fascia CF. The fibrils F shown in the figure have lost elasticity over time in this subject, resulting in downward tension at the dermis D, and dimpling DI at the epidermis E (i.e., at the skin surface).

As shown in FIG. 2, by targeting disruption via HIFU treatments 10 between the dermis D and the campers fascia CF, a percentage of the fibrils F supporting the dermis D may be severed or disrupted. The fibrils F may be severed by a moving transducer, a stationary transducer that is moved to multiple locations, or by the superheated pockets of liquid as described above. As discussed above, the amount of fibrils disrupted may be any percentage, but in embodiments is 30% or greater.

Severing the fibrils F allows the dermis D to be released or at least partially released from its connection to the campers fascia CF, as shown in FIG. 3, releasing the downward tension from the connection of the fibrils F to the campers fascia CF. At this point, there are two options, described below.

In first embodiments, methods are provided for disrupting the collagen fibrils F in a temporary manner, with the fibrils repairing and restoring during a wound healing process. As described in “Tissue repair and the dynamics of the extracellular matrix” (The International Journal of Biochemistry & Cell Biology, 36 (2004) 1031-1037), incorporated herein by reference, wound healing is a repair of tissue after injury where a provisional fibrin fibronectin matrix is contracted to bring margins of a wound together, leaving a collagen rich scar tissue. The contraction results in at least a temporary reduction of the overall length of natural collagen fibrils so that a single “strand” of collagen prior to treatment, is reduced length-wise shortly after treatment.

First, disruption occurs between the dermis D and the campers fascia CF via HIFU, as described above with reference to FIG. 2, with the disrupted fibrils shown in FIG. 3. For purposes of illustration, all fibrils in FIG. 3 are shown as disrupted, but as described above, a portion, such as 30% or greater, of the fibrils may be disrupted. For such disruption, the HIFU treatments are preferably targeted between these two layers so as to release the connection between these two layers by the fibrils, although targeting the HIFU just below these layers may have the same result due to the halo effect described earlier. Any form of disruption utilizing HIFU may provide the disruption effect.

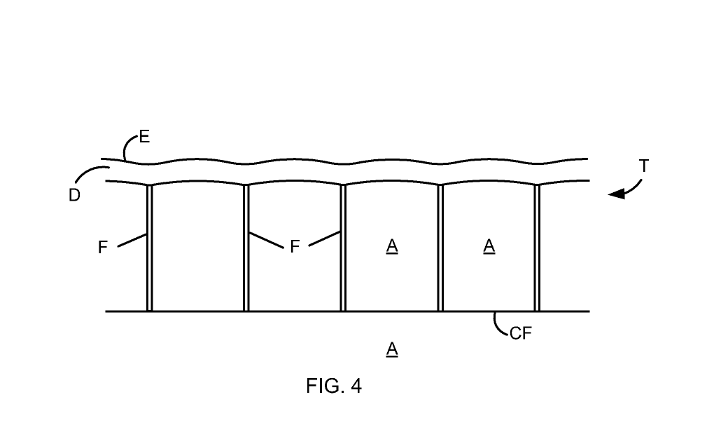

A wound healing process then occurs, resulting in the damaged or disrupted fibrils being replaced by stronger, more elastic fibrils, F, as shown in FIG. 4. These new fibrils F are more elastic and pull downward with less tension on the dermis D and the epidermis, resulting in more uniform pulling at the dermis D, and an appearance of less dimpling at the surface of the patient's skin, and thus less appearance of cellulite.

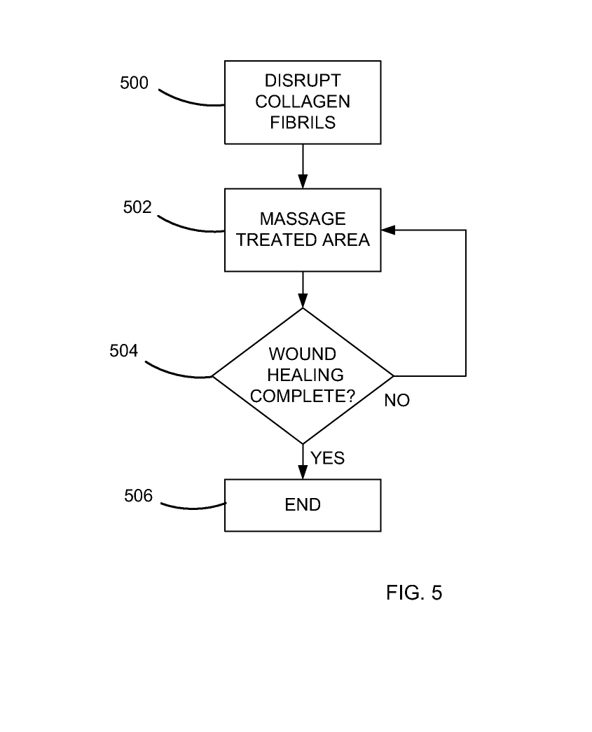

FIG. 5 shows a method of disrupting collagen fibrils F in accordance with additional embodiments. Beginning at 500, collagen fibrils are disrupted, as described above in connection with FIG. 2. At 502, after disruption of collagen fibrils F between the dermis D and the campers fascia CF (FIG. 2), the patient undergoes a massage in the treated area, typically shortly (e.g., several days) after the HIFU treatment and typically, subsequently massaged again several times over a period of days or weeks. The massage treatments stretches out weakened collagen fibrils allowing the skin to smooth out until the wound healing process replaces and remodels damaged and the disrupted fibrils F with new more elastic fibrils.

The wound healing process continually proceeds to a final remodeling state, so in embodiments, the patient has repeated massages, with delays between, over days until the wound healing process has remodeled and replaced the damaged or disrupted fibrils F with new more elastic fibrils. To this end, at 504, a determination is made whether the wound healing process is terminated, for example, by the lapse of a period of time determined through test procedures (e.g., a month). If the process has not completed, 504 branches back to 502, where another massage is given, for example a couple of days after the first. The process then proceeds back to 504, and a loop is maintained until the wound healing process is terminated. When the wound healing process has been substantially completed by the patient's body, the process ends at 506.

In embodiments, the period between massages is sufficiently short to prevent damaged or disrupted fibrils F from contracting, and lasts until the damaged and disrupted fibrils F have been replaced with new more elastic fibrils. These new more elastic fibrils provide less tension to the dermis D, smoothing the dimples DI at the patient's skin.

In additional embodiments, there are provided a method of and device for non-invasively contracting collagen fibrils in animal tissue. The method comprises presenting of a HIFU apparatus on a skin surface, coupling the HIFU apparatus to the skin surface to maximize energy coupling from the HIFU apparatus through the skin surface and activating the HIFU apparatus to deposit an average fluence (EF) value of at least 35 J/cm² such that collagen fibrils below the dermis are reduced at least 30% in length. In embodiments, the method occurs between the dermis and campers fascia. The device includes a HIFU apparatus programmed or configured to, or programmable to, provide such methods.

The collagen fibrils within the animal tissue are generally within or very close to the focal zone of a HIFU transducer incorporated into the apparatus, although it is not necessarily the case that the HIFU transducer is within an apparatus. In an embodiment the HIFU transducer may be “naked” without any structural elements surrounding it.

The peak intensity and average fluence of the HIFU apparatus can be used to produce the contraction (or denaturing) of collagen fibrils. Denaturing may occur due to wound healing that takes place as a result of the application of sufficient HIFU fluence, as described above. In general, the fibrils are heated so as to damage the fibrils, but not disrupt the fibrils. To heal after damage, the fibrils undergo an immediate contraction process, whereby damage, typically heat, causes bonds between strands of collagen fibrils forming a crystalline structure to break and form a more amorphous shorter and thicker structure. Immediate contraction results in at least a temporary reduction of the overall length of natural collagen fibrils so that a single “strand” of collagen prior to treatment, is reduced in length after treatment. Immediate reactions caused by heat are discussed, for example, in “Near Painless, Nonablative, Immediate Skin Contraction Induced by Low-Fluence Irradiation with New Infrared Device: A Report of 25 Patients,” Ruiz Esparza, MD, Dermatol Surg 32:601-610 (2006), incorporated herein by reference. The report teaches that collagen contraction is immediate upon application of heat and results in the breakage of hydrogen bonds holding the collagen fibers in a crystalline structure of triple helices of protein chains. It proceeds to teach that heated collagen transforms from the crystalline triple heated structure to an amorphous, random-coil structure through the breakage of the hydrogen bonds. This creates a thickening and shortening of the collagen fibers as the chains fold and assume a more stable configuration. This effect can persist for months and may be accompanied by a longer term wound-healing process.

The peak fluence may be the same as the average fluence, or the peak fluence may be higher. In an aspect of the invention, the average fluence may vary between 35 and 460 J/cm². In another aspect the average fluence is 100 J/cm². In still another aspect the average fluence is 174 J/cm², and in yet another aspect the average fluence is 265 J/cm². Average fluence variations such as these embodiments, and other embodiments as may be determined without undue experimentation allow an operator to optimize the peak intensity output and average fluence values to achieve a desired clinical result in a target tissue.

In various embodiments, the target tissue(s) may be human adipose tissue and/or human subcutaneous tissue. Applicant possesses data showing the disruption of collagen fibrils in both humans and animal studies. While data exists for both models (human and porcine) the larger body of data is produced in human clinical trials.

As in other embodiments, the efficiency of denaturing collagen fibrils may be enhanced by moving the HIFU apparatus over the skin surface. The denaturing of collagen fibrils varies by patient, based on various factors, such as the morphology of the target tissue, the EF value used, and variations in tissue from patient to patient. In one aspect the reduction in collagen fibrils may be 30% in length. In another aspect the reduction may be 40%. In still another aspect, the reduction may be 50% in length.

Measurement of the reduction in length is made using histology data, and can be evaluated from porcine model studies, abdominoplasty analysis in humans or removal of the targeted tissue following the application of HIFU energy at the desired EF values.

In addition to denaturing collagen, the method may induce collagen fibril disruption and/or adipose tissue disruption.

In another embodiment, there is a method of measuring skin tone improvement. The method comprises locating a region of human tissue beneath a skin surface and determining an initial condition for the skin surface. Then injuring the region of human tissue using a HIFU apparatus such that a volume of disrupted adipose cells and denatured and/or disrupted collagen fibrils are produced and determining one or more post operative condition(s) for the skin surface after the injury to the region of human tissue has been created. In one aspect, the method includes identifying a patient that is seeking improvement in terms of cellulite and/or loose skin and applying any of the methods described herein to such a patient. In a further facet of such an aspect, the invention provides a step of monitoring the patient for improvement in terms of skin elasticity/tightness and/or in terms of cellulite data (such as area and/or amount and/or depth of dimpling/dimples, etc.). Methods may further include the step of re-treatment if desired results are not obtained and/or combining any of the methods described herein with other known methods of enhancing or maintaining such conditions (e.g., loose skin and/or cellulite).

In one aspect, the determining at the post operative conditions may occur after the injury to the region of tissue has naturally resolved. Post operative condition evaluation may indicate the improved appearance of cellulite, the reduction of wrinkles in the skin (as measured using a wrinkle measuring scale such as Fitzpatrick or Glogau classification). In another aspect, the general skin tone can be measured and compared in the evaluation of the skin prior to the activation of the HIFU apparatus, and after. Post operative measurement may be made any number of times, and at any time interval after the HIFU apparatus treatment.

Collagen denaturing can occur at temperatures above, e.g., 37° C. However treated collagen at temperatures close to normal body temperature may recover, relax and resume their normal length. Collagen denaturing by application of HIFU in methods of this invention can be obtained under any suitable conditions. In an embodiment, collagen in the treatment zone is exposed to temperatures above 37° C. In another embodiment collagen fibrils in the treatment zone are exposed to temperatures above 46° C. and in still another embodiment, the collagen fibrils are heated to a temperature above 56° C. The higher the temperature the collagen fibrils are exposed to, the shorter the length of time needed to achieve the desired effect (permanent collagen denaturing for contraction of remodeled collagen fibrils). When the exposure is at 46° C. the collagen fibrils need to be incubated at that temperature for several minutes, however exposure of collagen fibrils to temperatures near or above 56° C. may be done in less than a few seconds.

In an embodiment, adipose tissue is heated using HIFU energy so the temperature in the lesion field is raised as high as practical and as fast as possible. Parameters of the HIFU transducer may be adjusted to produce the desired fast heating needed to destroy adipose tissue and denature collagen fibrils. The fast heating is balanced with the volume and dimensions of the adipose tissue to be treated. The longer the transducer remains active on one location, the larger the halo field. To protect adjacent tissue, careful planning of tissue treatment is desired so the moving of the HIFU transducer and the applying of therapeutic ultrasound energy do not produce lesion or halo fields which extend beyond the dimensions of the target tissue volume.

Additional parameters that affect the size of the lesion and halo fields are those parameters electronically controlled through the transducer, and parameters of the transducer itself. These parameters include (but are not limited to) power, frequency, duty cycle, focus, aperture size (of transducer), and pulse repetition frequency.

In another embodiment, the lesion field may be controlled through modifying the aperture and duty cycle of a mechanically focused ultrasound transducer, or an array transducer with the proper electronic controller.

Alternatively, the lesion and halo fields may be maximized by permitting the HIFU transducer to produce contiguous lesion fields and cooperative halo fields. In this embodiment, the energy required to produce cellular necrosis and collagen contraction is lessened due to the co-operative effect of having the transducer operate in narrowly spaced treatment lines and in rapid succession of laying down treatment lines near each other in both time and space. Movement of the transducer is desirably machine controlled for uniformity and simultaneous control of the transducer. The transducer can treat patient tissue volume by moving over the surface of the tissue volume in any variety of patterns including, but not limited to, spiral, raster scan, or patterned. Thermal cooperation can be maximized by delivering the ultrasound energy as a contiguous lesion field within a treatment site. Careful planning and consideration in the applying of ultrasound energy in the methods described herein can produce the desired volume of tissue modification in both the amount of adipose tissue destroyed, and collagen denatured and/or disrupted.

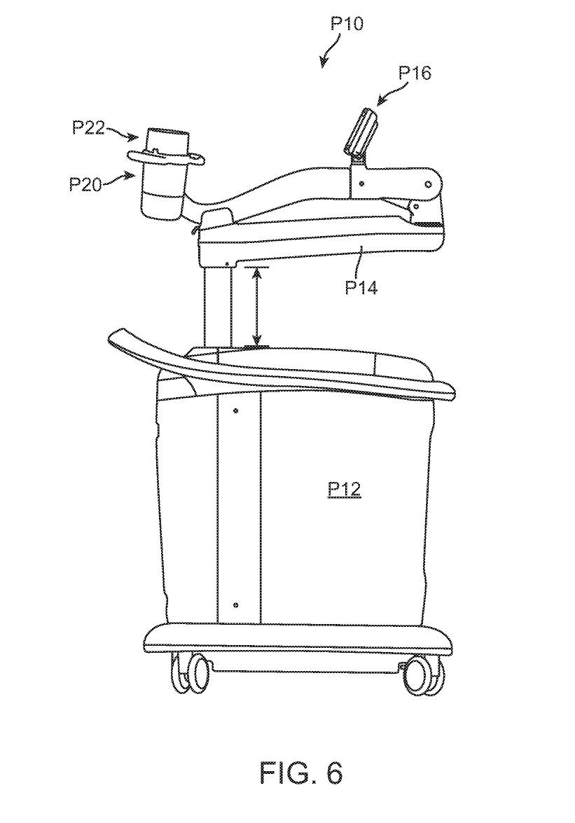

A HIFU device P10 that may be used to perform the acts described herein is shown in FIG. 6. The system P10 has a cart base P12 with a mechanical arm P14 supporting a therapy head P20 with a removable cap P22. A transducer (not shown, but known) is located in the therapy head P20. As is known, an operator may manipulate the therapy head into contact with a patient and operate the system P10 to deliver HIFU treatments into a patient. In embodiments, such a device P10 is programmed, programmable, or configured to provide such treatments in accordance with the procedures and parameters described herein. Details of such a system can be found in US patent application number 2009/0240146, and an alternate embodiment can be found in US patent application number 2011/0077514, both of which are incorporated herein by reference.

In embodiments, the system P10 may be programmed to provide the functions herein, or may be programmed to receive input from a user so that it may provide such functions. To this end, the system P10 may include a computer system or other controller to permit such programming.

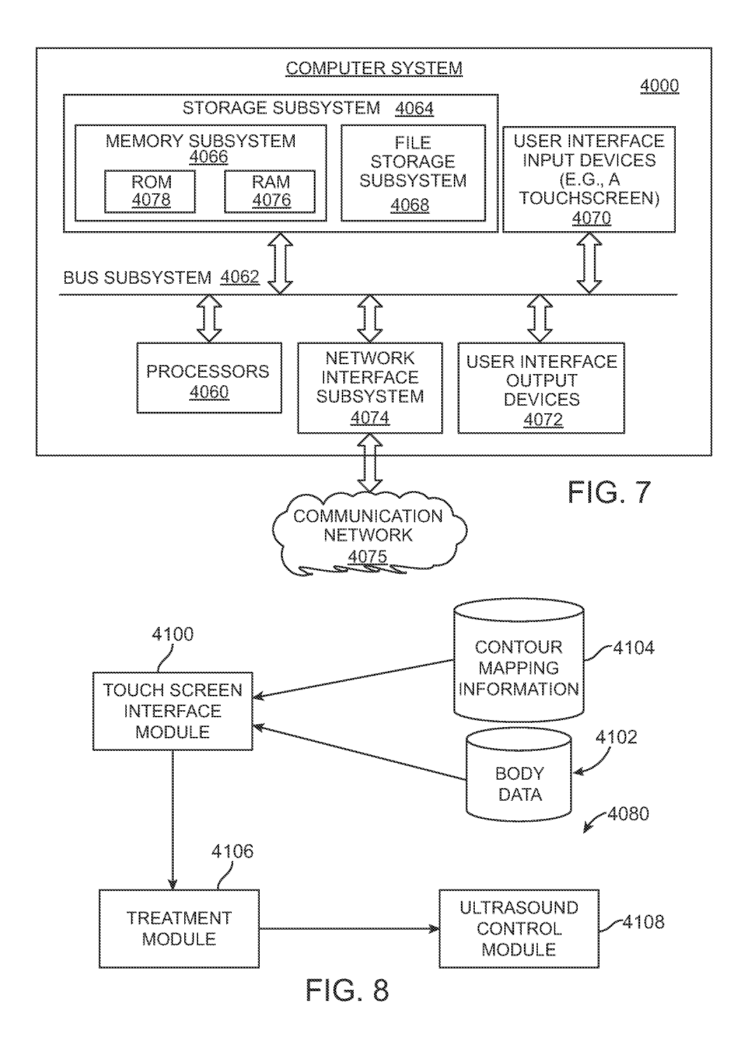

FIG. 7 is a simplified block diagram of an example computer system 4000 that may provide such functions in accordance with embodiments. The computer system typically includes at least one processor 4060 which communicates with a number of peripheral devices via a bus subsystem 4062. These peripheral devices may include a storage subsystem 4064, comprising a memory subsystem 4066 and a file storage subsystem 4068, user interface input devices 4070, user interface output devices 4072, and a network interface subsystem 4074. Network interface subsystem 4074 provides an interface to a communication network 4075 for communication with other imaging devices, databases, or the like.

The processor 4060 performs the operation of the computer systems 4000 using execution instructions stored in the memory subsystem 4066 in conjunction with any data input from an operator, if provided. Such data can, for example, be input through user interface input devices 4070, such as the graphical user interface. Thus, processor 4060 can include an execution area into which execution instructions are loaded from memory. These execution instructions will then cause processor 4060 to send commands to the computer system 4000, which in turn control the operation of the ultrasound control electronics. Although described as a “processor” in this disclosure and throughout the claims, the functions of the processor may be performed by multiple processors in one computer or distributed over several computers.

User interface input devices 4070 may include a keyboard, pointing devices such as a mouse, trackball, touch pad, or graphics tablet, a scanner, foot pedals, a joystick, a touch screen incorporated into the display, audio input devices such as voice recognition systems, microphones, and other types of input devices. In general, use of the term “input device” is intended to include a variety of conventional and proprietary devices and ways to input information into the computer system. Such input devices will often be used to download a computer executable code from a computer network or a tangible storage media embodying steps or programming instructions for any of the methods of the present invention.

User interface output devices 4072 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide non-visual display such as via audio output devices. In general, use of the term “output device” is intended to include a variety of conventional and proprietary devices and ways to output information from the computer system to a user.

Storage subsystem 4064 stores the basic programming and data constructs that provide the functionality of the various embodiments. For example, database and modules implementing the functionality of embodiments described herein may be stored in storage subsystem 4064. These software modules are generally executed by processor 4060. In a distributed environment, the software modules may be stored in a memory of a plurality of computer systems and executed by processors of the plurality of computer systems. Storage subsystem 4064 typically comprises memory subsystem 4066 and file storage subsystem 4068.

Memory subsystem 4066 typically includes a number of memories including a main random access memory (RAM) 4076 for storage of instructions and data during program execution and a read only memory (ROM) 4078 in which fixed instructions are stored. File storage subsystem 4068 provides persistent (non-volatile) storage for program and data files, and may include a hard disk drive, re-writable non-volatile memory chips (such as Flash memory), a floppy disk drive along with associated removable media, a Compact Digital Read Only Memory (CD-ROM) drive, an optical drive, DVD, CD-R, CD-RW, or removable media cartridges or disks. One or more of the drives may be located at remote locations on other connected computers at other sites coupled to the computer system. The databases and modules implementing the functionality of the present invention may also be stored by file storage subsystem 4068. The file storage subsystem may have directory and file descriptions for accessing the files, or it may store data without descriptions and rely on the databases and modules of the system to locate the data.

Bus subsystem 4062 provides a mechanism for letting the various components and subsystems of the computer system communicate with each other as intended. The various subsystems and components of the computer system need not be at the same physical location but may be distributed at various locations within a distributed network. Although bus subsystem 4062 is shown schematically as a single bus, alternate embodiments of the bus subsystem may utilize multiple busses.

The computer system 4000 itself can be of varying types including a personal computer, a portable computer, a workstation, a computer terminal, a network computer, a module in a circuit board, a mainframe, or any other data processing system. Due to the ever-changing nature of computers and networks, the description of the computer system depicted in FIG. 7 is intended only as a specific example for purposes of illustrating one embodiment. Many other configurations of the computer system are possible having more or less components than the computer system depicted in FIG. 48.

FIG. 8 schematically illustrates a plurality of modules 4080 that may carry out embodiments. The modules 4080 may be software modules, hardware modules, or a combination thereof. If the modules are software modules, the modules will be embodied on a computer readable medium and processed by a processor 4060 in any of computer systems of the present invention.

A first module is a touch screen interface module 4100. The touch screen interface module receives data from the touch screen, e.g., the user interface input device 4070, as described above. In addition, in embodiments, the touch screen interface module may be configured to receive body data 4102 and/or contour/mapping information 4104.

Information from the touch screen interface module is forwarded to a treatment module 4106. The treatment module 4106 generates treatment information and forwards that information to an ultrasound control module 4108, which in turn controls the ultrasound electronics for the device.

The modules 4080 are designed so that an operator may enter information into a touch screen interface, which is in turn received by the touch screen interface module 4100. The touch screen can detect menu selections and freehand drawings or other contact made with the touch screen made using either a stylus or a finger of the user.

As an alternative, the computer system 4000 may be configured to be programmed without body data 4102 or contour information 4104. In addition, the computer may be programmed to provide treatment at a predefined depth that is consistent with a location between the dermis and campers fascia of a patient.

Review of the material disclosed herein will provide one skilled in the art with numerous alternative methods of accomplishing the desired objectives described, as well as methods not specifically mentioned herein. The description provided should be taken as illustrative and non-limiting, thus the present embodiments as well as alternative embodiments and equivalents are intended to be captured by the accompanying claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method of non-invasively disrupting collagen fibrils in animal tissue, the method comprising:
presenting of a HIFU apparatus on a skin surface of a patient;
coupling said HIFU apparatus to said skin surface to maximize energy coupling from said HIFU apparatus through said skin surface;
activating said HIFU apparatus to deposit an average fluence (EF) value of at least 35 J/cm² such that collagen fibrils below the dermis of the patient are at least 30% disrupted.

2. The method of claim 1, wherein the average EF value is at least 100 J/cm².

3. The method of claim 1, wherein the average EF value is at least 174 J/cm².

4. The method of claim 1, wherein the average EF value is at least 265 J/cm².

5. The method of claim 1, wherein said animal tissue comprises human adipose tissue.

6. The method of claim 1, wherein said animal tissue comprises human subcutaneous tissue.

7. The method of claim 1, wherein said fluence is deposited between the dermis and campers fascia of the patient.

8. The method of claim 1, wherein the animal tissue is in a human patient (i.e., the method is applied in vivo).

9. The method of claim 1, further comprising moving said energy applicator over the skin surface while depositing said fluence.

10. The method of claim 1, wherein the collagen fibrils are at least 50% disrupted.

11. The method of claim 1, wherein the collagen fibrils are at least 65% disrupted.

12. The method of claim 1, wherein the collagen fibrils are at least 75% disrupted.

13. The method of claim 1, wherein the collagen fibrils are at least 80% disrupted.

14. The method of claim 1, further comprising denaturing one or more collagen fibrils as a result of depositing said fluence.

15. The method of claim 1, further comprising destroying one or more adipose cells when said HIFU apparatus is activated.

16. The method of claim 1, further comprising massaging the area treated by said deposit of fluence.

17. The method of claim 16, further comprising regularly messaging said treatment area until a wound healing process for the disrupted fibrils is substantially complete.

18. A method of non-invasively contracting collagen fibrils in animal tissue, the method comprising:
presenting of a HIFU apparatus on a skin surface of a patient;
coupling said HIFU apparatus to said skin surface to maximize energy coupling from said HIFU apparatus through said skin surface;
activating said HIFU apparatus to deposit an average fluence (EF) value of at least 35 J/cm² such that collagen fibrils below a dermis of the patient are reduced at least 30% in length.

19. The method of claim 18, wherein the average EF value is at least 100 J/cm².

20. The method of claim 18, wherein the average EF value is at least 174 J/cm².

21. The method of claim 18, wherein the average EF value is at least 265 J/cm².

22. The method of claim 18, wherein said animal tissue comprises human adipose tissue.

23. The method of claim 18, wherein said animal tissue comprises human subcutaneous tissue.

24. The method of claim 18, wherein said animal tissue comprises human tissue between the dermis and campers fascia of the human.

25. The method of claim 18, further comprising moving said energy applicator over the skin surface.

26. The method of claim 18, wherein the collagen fibrils are reduced at least 40% in length.

27. The method of claim 18, wherein the collagen fibrils are reduced at least 50% in length.

28. The method of claim 18, further comprising disrupting one or more collagen fibrils when said HIFU apparatus is activated.

29. The method of claim 18, further comprising destroying one or more adipose cells when said HIFU apparatus is activated.

30. A method of measuring skin tone improvement, the method comprising:
locating a region of human tissue beneath a skin surface;
determining an initial condition for said skin surface;
injuring said region of human tissue using a HIFU apparatus such that a volume of disrupted adipose cells and denatured and/or disrupted collagen fibrils are produced;
allowing said injured region of human tissue to resolve; and
determining one or more post operative condition(s) for said skin surface after the injury to said region of human tissue has been created.

31. The method of claim 30, wherein determining at least one of said post operative conditions occurs after the injury to the region of tissue has naturally resolved.

32. A device for non-invasively disrupting collagen fibrils in animal tissue, the device comprising:
a HIFU apparatus designed to be coupled to a skin surface of a patient; and
a computer system configured to activate said HIFU apparatus, when the HIFU apparatus is against the skin of a patient, to:
deposit an average fluence (EF) value of at least 35 J/cm² so as to disrupt collagen fibrils; and
focus a focal point of fluence from the HIFU apparatus below the dermis of the patient.

33. The device of claim 32, wherein the average EF value is at least 100 J/cm².

34. The device of claim 32, wherein the average EF value is at least 174 J/cm².

35. The device of claim 32, wherein the average EF value is at least 265 J/cm².

36. The device of claim 32, wherein the focal point is directed between the dermis and campers fascia of the patient.

37. The device of claim 32, wherein the HIFU apparatus and the computer system are designed such that the HIFU apparatus is movable over the skin surface while depositing said fluence.

38. The device of claim 32, wherein the fluence is sufficient such that the collagen fibrils are at least 50% disrupted.

39. The device of claim 32, wherein the fluence is sufficient such that the collagen fibrils are at least 65% disrupted.

40. The device of claim 32, wherein the fluence is sufficient such that the collagen fibrils are at least 75% disrupted.

41. The device of claim 32, wherein the fluence is sufficient such that the collagen fibrils are at least 80% disrupted.

42. A device for non-invasively contracting collagen fibrils in animal tissue, the device comprising:
a HIFU apparatus designed to be coupled to a skin surface of a patient;
a computer system configured to activate said HIFU apparatus, when the HIFU apparatus is against the skin of a patient, to:
deposit an average fluence (EF) value of at least 35 J/cm² so as to reduce collagen fibrils at least 30% in length; and
focus a focal point of fluence from the HIFU apparatus below the dermis of the patient.

43. The device of claim 42, wherein the average EF value is at least 100 J/cm².

44. The device of claim 42, wherein the average EF value is at least 174 J/cm².

45. The device of claim 42, wherein the average EF value is at least 265 J/cm².

46. The device of claim 42, wherein the HIFU apparatus and the computer system are designed such that the HIFU apparatus is movable over the skin surface while depositing said fluence.

47. The device of claim 42, wherein the fluence is sufficient such that the collagen fibrils are reduced at least 40% in length.

48. The device of claim 42, wherein the fluence is sufficient such that the collagen fibrils are reduced at least 50% in length.

49. The device of claim 42, wherein the focal point is directed between the dermis and campers fascia of the patient.
